(19) United States
(12) Patent Application Publication
(10) Pub. No.: US 2003/0212371 A1
(43) Pub. Date: Nov. 13, 2003

(54) Disposable safety syringe

(76) Inventors: Martin E. Smith, Camarillo, CA (US); Thomas R. Coughlin JR., Tulsa, OK (US); Earl R. Boyer, Simi Valley, CA (US); Sturart J. Daley, Camarillo, CA (US)

Correspondence Address: Loren G. Helmreich, Browning Bushman PC, Suite 1800, 5718 Westhelmer Road, Houston, TX, 77057-5771, US

(21) Appl. No.: 10/143,290

(22) Filed: May 10, 2002

(51) Int. Cl. A61M 5/315

(52) U.S. Cl. 604/229; 604/240; 604/110

(57) ABSTRACT

A safety syringe 10 includes a generally tubular body 14 having a needle end 18 and plunger end 22, needle 38 and retractable needle seat 30, two-way valve 36, plunger 42, stopper 62, and rear plunger seal 44. The plunger may be selectively engageable with both the stopper and the retractable needle seat. During use, the plunger may be moved to capture the stopper. The user then moves the plunger and the captured stopper toward the needle end to a displaced position corresponding to the desired volume of fluid to be withdrawn. A vacuum is created between the stopper and rear plunger seal, and helps to draw fluid into the tubular body. At the conclusion of the patient injection stroke, the plunger engages the needle seat, and the vacuum between the stopper and rear plunger seal retracts the needle safely into the tubular body. The two-way valve controls flow into and out of the body during use.

### FIELD OF THE INVENTION

The present invention relates generally to syringes and, more particularly, to syringes having retractable needles. The invention specifically relates to a disposable syringe which utilizes a vacuum to automatically retract the needle into the syringe body and thereby prevent the healthcare practitioner from getting stuck by the needle.

### BACKGROUND OF THE INVENTION

Hypodermic syringes provide an effective, reliable, and inexpensive way to inject a measured quantity of medicine below the skin. Syringes typically have exposed needles, however, and the ease by which a needle may pierce the skin creates a hazard that the healthcare worker may accidentally be stuck with a needle. The resulting injury could be as simple as a minor skin laceration, or as deadly as an infection from a virus in the patient's blood.

Because syringes are routinely used worldwide, occasional injuries are inevitable when using conventional syringes. Healthcare practitioners are exposed to this danger in routine medical practice. Diabetics, people with arthritis, and others who self-administer daily injections are at risk, as are members of their household. After disposal, conventional syringes may continue to pose a risk to sanitation workers and anyone else who comes in contact with landfills and waste management processes. Some syringes will undoubtedly be disposed of or handled improperly prior to disposal, increasing the chance of injury. Despite their utility, conventional syringes thus clearly pose a danger to healthcare practitioners.

The prior art discloses methods of reducing or eliminating the dangers associated with exposed syringe needles. U.S. Pat. No. 4,908,022 describes a disposable safety syringe having a retractable needle. U.S. Pat. No. 5,885,257 describes a syringe having a spring-loaded, automatically retractable needle. U.S. Pat. No. 5,000,736 describes a syringe having a sealed tubular plunger from which air has been evacuated and a needle releasably attached to the distal end. After the patient is injected, the plunger seal is ruptured and the differential pressure between the vacuum and ambient air causes the needle to retract safely within the syringe body. U.S. Pat. No. 6,193,695 describes a syringe having a sealed portion between the plunger and end cap. As the plunger is pulled away from the needle to fill the syringe, a one-way valve in the sealed portion opens, allowing air to be expelled from the sealed portion. During injection, the plunger is moved toward the needle to expel its contents, the valve closes, and the pressure in the sealed portion decreases. At the end of its stroke, the plunger captures the needle, and the relatively low pressure in the sealed portion causes the plunger and needle to retract into the syringe body. Other patents of interest include U.S. Pat. Nos. 4,425,120; 4,643,200; 4,675,005; 4,692,156; 4,747,830; 4,816,022; and 4,790,822.

Although the prior art has addressed many of the safety problems related to conventional hypodermic syringes, numerous shortcomings remain relating to the cost of manufacturing and the safe use of syringes with retractable needles (safety syringes). Some safety syringes require storage of potential energy, which may be unreliable. For example, sealed vacuum chambers are prone to leakage when the syringe is stored for an extended period. Other safety syringes may have needles which retract a limited distance, with the retracted needle remaining dangerously close to the syringe body opening. Some syringes are shipped and stored with plungers fully extended, increasing their packaged size with a corresponding decrease in the efficiency of shipping and storage. Syringes which utilize springs are costly, and require additional seals to prevent contamination of the fluid drawn into the body of the syringe. Further shortcomings exist in the prior art with regard to manufacturing cost, ease of use, and reliability of safety syringes. A reliable syringe which automatically retracts the needle into the syringe body is sought which overcomes the disadvantages of the prior art.

The present invention surpasses the prior art, offering an improved safety syringe that is both reliable and economical. The retractable-needle or safety syringe of the present invention is relatively simple and convenient to operate.

### SUMMARY OF THE INVENTION

The present invention is directed to a safety syringe that retracts its needle into a syringe body to prevent the healthcare practitioner from accidentally getting stuck by the exposed needle. The retractable needle protects various people, including healthcare workers and their patients, and sanitation workers involved with disposal of medical waste. The invention may prevent or reduce injuries ranging from minor skin lacerations to serious contamination by medications, germs, or viruses. The syringe preferably is a disposable, single-use device, and may be available in various sizes and shapes. A syringe according to this invention may also be used in non-medical applications, such as chemical handling processes.

It is an object of the present invention to provide an improved vacuum operated, retractable-needle (safety) syringe. A preferred embodiment includes a selectively retractable needle assembly comprising a needle seat for supporting a needle, and a generally tubular body that serves as a reservoir both for injectable or withdrawn fluids and for a vacuum chamber. A stopper may be selectively engaged by a plunger and seals against the inside of the tubular body to facilitate withdrawing fluids and creating the vacuum. The plunger is axially moved both to draw fluid into the syringe, expel fluid from the syringe into the patient, and create the vacuum within the tubular body. A two-way valve regulates the flow of fluid and air flowing into and out of the tubular body during use. A ridge or other connector on the plunger may selectively engage the stopper so that the stopper and plunger therefrom move as an assembly. A latch on the plunger or the stopper selectively engages the needle seat to withdraw the needle into the generally tubular body due to created vacuum.

The syringe may be distributed and stored in a relatively compact packaged configuration, with the plunger substantially retracted into the tubular body. A seal between the plunger and tubular body (plunger seal) allows the plunger to be moved axially within the generally tubular body while preventing air from passing into or out of the body through the rear end of the plunger.

To use the syringe, the healthcare practitioner may first move the plunger toward the plunger end to capture the stopper. With the stopper captured, the plunger and stopper may now move as an assembly within the tubular body. The practitioner then forces the plunger axially toward the needle end, which increases the volume of the sealed portion of the tubular body between the stopper and plunger seal. Because air cannot enter, pressure decreases and a vacuum is created in the sealed portion between the stopper and the rear seal. Since the opposing side of the plunger is at atmospheric pressure, an axial force due to the difference in the fluid pressure is directed toward the plunger end of the tubular body, with a substantially equal and opposite force exerted by the practitioner on the plunger. The tubular body may have a reverse graduation which the practitioner may use as a reference to axially move the plunger a distance corresponding to the volume of fluid to be withdrawn from the vial.

To fill the tubular body, the practitioner may first insert the needle into a vial or other fluid source and move the plunger axially toward the needle end of the tubular body as described above, thereby pressurizing the vial. The practitioner may then move the plunger axially toward the plunger end, assisted by the vacuum, to draw fluid into the tubular body. The practitioner may then aspirate the syringe in a conventional manner, eliminating air and any excess liquid. To inject the patient, the practitioner inserts the needle under the skin and forces the plunger toward the needle end of the body to expel the fluid, thereby again creating a vacuum in the sealed portion of the tubular body. At the end of the plunger injection stroke, a latch on the plunger or on the stopper engages the needle seat, so that the plunger, stopper, and retractable needle assembly now move as an assembly with respect to the tubular body. The practitioner may then release the plunger, and the vacuum in the sealed portion of the tubular body automatically moves the plunger toward the plunger end, retracting the needle safely within the syringe body.

It is an object of this invention to provide an improved safety syringe. The safety syringe may operate more reliably and consistently than other safety syringes. Because the vacuum which assists plunger movement is created during use, there is no need for a potential energy source which may degrade or fail prior to use. A related object of the invention is to provide an improved safety syringe which uses a vacuum within the generally tubular body of the syringe created by movement of the plunger to withdraw the needle seat and the attached needle into the generally tubular body.

Another object of the invention is to provide an improved method of using a syringe of the type with a needle seat sealingly engaged within the tubular body for supporting a needle, with the needle seat being releasably retained on the tubular body in an initial position. Movement of the plunger and an axially connected stopper creates a vacuum between a rear plunger seal and the stopper. A practitioner may apply a first axial force to move the plunger to a displaced position, then insert the needle into the liquid source and withdraw a selected volume of liquid into the tubular body. After aspirating the air, the needle is inserted into a fluid repository, such as a patient, and a second axial force on the plunger is used to discharge liquid from the needle and simultaneously create a vacuum within the tubular body. At the end of the injection stroke, a seat/plunger latch connects the needle seat with the plunger, or optionally with the stopper. By relaxing the second force, the needle seat is disengaged from the tubular body and the plunger and connected needle seat and needle are moved to the retracted position within the tubular body.

A feature of the invention is a syringe which uses a two-way valve between the needle seat and the generally tubular body to control the flow of air and liquid into and out of the body. During use, this two-way normally closed valve also may partially regulate the force required to move the plunger.

Another feature of the invention is the use of a stopper/plunger latch for selectively axially connecting the stopper to the plunger by axial movement of the plunger with respect to the tubular body. This feature allows the syringe to have a compact size for shipment and storage. The stopper/plunger latch may include circumferentially spaced protrusions extending radially outward from a cylindrical surface of the plunger.

Another feature of the invention is that a “reverse” graduation may be provided on the tubular body increasing from the plunger end toward the needle end. The reverse graduations are used by the practitioner to move the plunger to a selected position for withdrawing the desired quantity of liquid into the syringe.

A significant advantage of the present invention is that the syringe may be manufactured at a relatively low cost, and accordingly the syringe preferably is a disposable single-use device.

Another significant advantage of the present invention is that the safety syringe may be easily and safely operated by the practitioner.

These and further objects, features, and advantages of the present invention will become apparent from the following detailed description, wherein reference is made to the figures in the accompanying drawings.

### BRIEF DESCRIPTION OF THE DRAWINGS

FIG. 1 illustrates in cross-section an embodiment of the safety syringe according to the present invention in an initial, packaged configuration.

FIG. 2 is an enlarged cross-sectional view of a portion of the syringe shown in FIG. 1.

FIG. 3 shows the plunger in a fully retracted position engaged with the stopper.

FIG. 4 illustrates in greater detail the plunger end of the cylinder body opposite the needle end, the stopper and the rear plunger seal.

FIG. 5 shows the syringe with the needle tip in a vial.

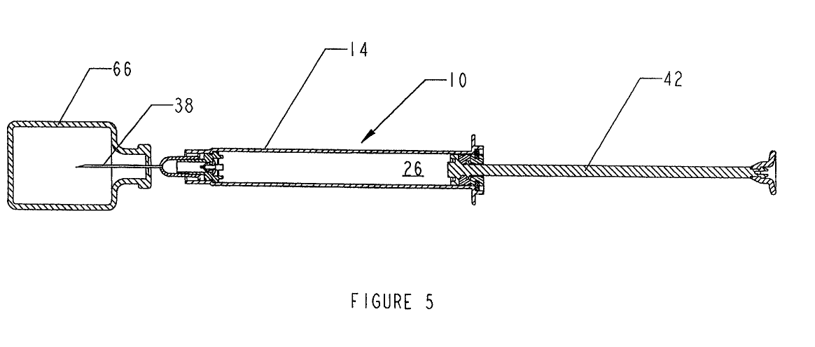

FIG. 6 shows in greater detail the needle end of the syringe and the retractable needle seat.

FIG. 7 shows the syringe after the plunger has been moved from an initial position to a selected, displaced position.

FIG. 8 shows the sealed vacuum portion between the stopper engaged with the plunger and the rear plunger seal.

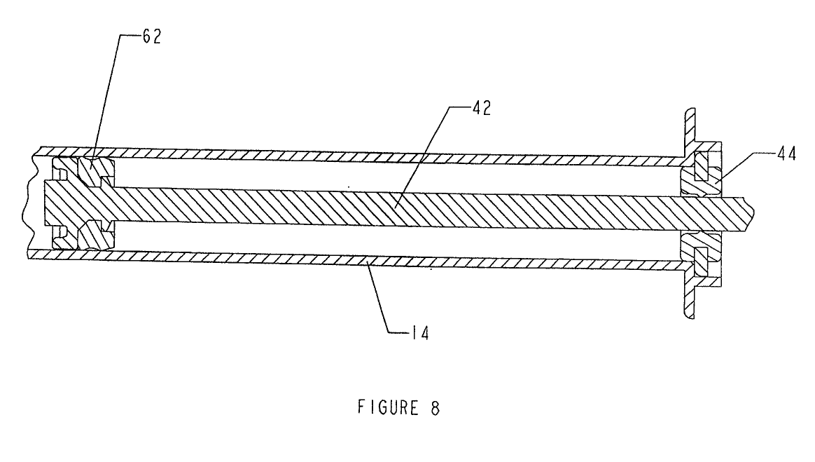

FIG. 9 shows in greater detail the two-way valve in the needle end of the tubular body.

FIG. 10 shows the syringe with its needle in a fully retracted position after use.

FIG. 11 is a pictorial view of the generally tubular body.

FIG. 12 is a pictorial view of the plunger.

FIG. 13 is a pictorial view of the needle seat.

FIG. 14 is a pictorial view of the two-way valve.

### DETAILED DESCRIPTION OF PREFERRED EMBODIMENTS

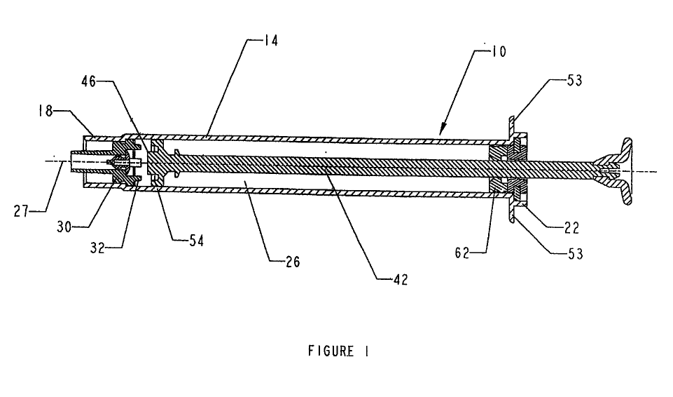

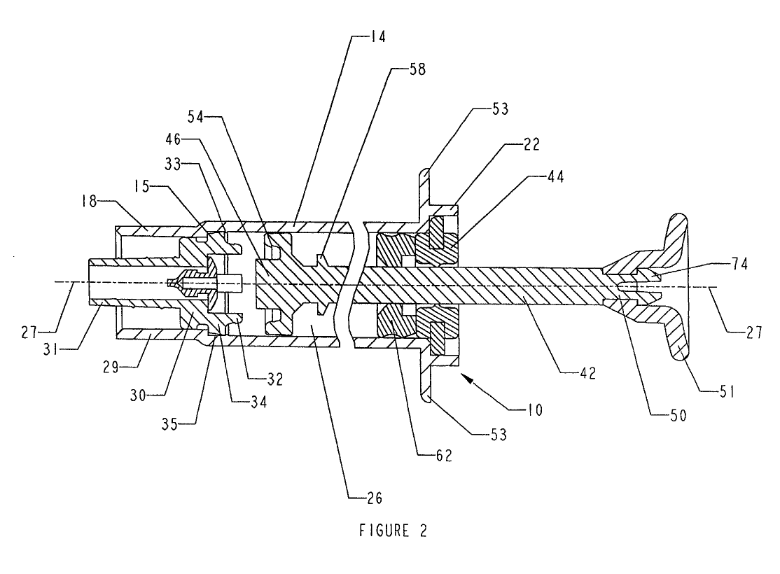

FIG. 1 shows a syringe 10 in an initial packaged configuration for shipment and storage, with greater detail shown in FIG. 2. A generally tubular body 14, which may house medicine or other liquid during use of the syringe 10, has a needle end 18, an opposing plunger end 22, a cylindrical throughbore 26, and a central axis 27. The tubular body 14 is preferably plastic, but may comprise other nonporous or leak proof materials.

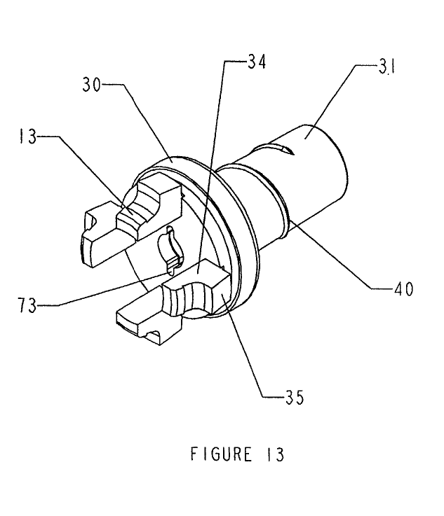

As shown in FIG. 2, a needle seat 30 is received within the throughbore 26 and at least partially within the reduced diameter portion 29 at the needle end 18 of the tubular body 14. Prior to use, the needle seat 30 is axially and sealingly attached to the tubular body 14, held in an initial retained position by a seta/body retainer 33 on the tubular body 14, which may be a generally annular bead. The radially outer portion 34 of the needle seat 30 is captured between the bead 33 and body stop 15, thereby securing the needle seat to the body 14. The radially outer portion 34 may comprise a plurality of circumferentially spaced tabs, as shown in FIG. 13. The radially outer surface 35 of each tab 34 is preferably tapered. The needle seat 30 is configured for release from the body 14, as discussed below.

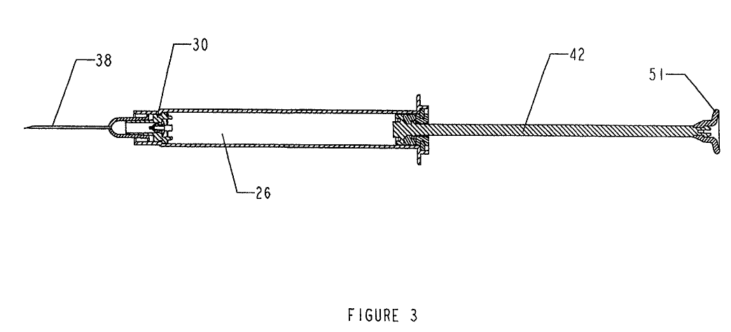

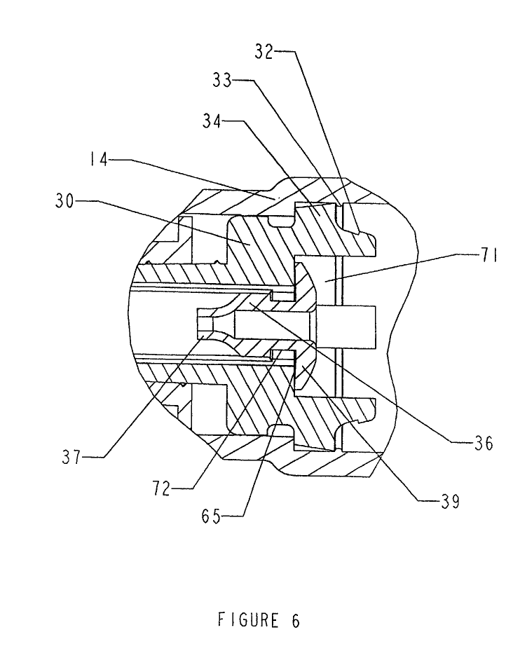

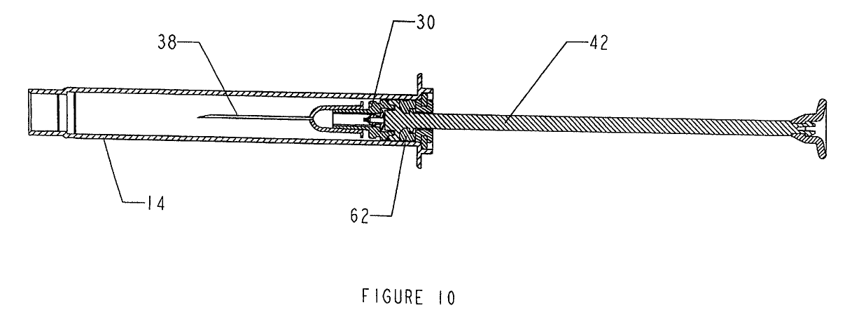

The needle seat 30 supports a needle 38 (see FIG. 3) which may pierce a layer of skin to transport medicine or other fluid subdermally. The needle seat end portion 31 as shown in FIG. 2 may be configured for attachment to a conventional needle/hub by a quarter-turn “spiral-lock” conventionally used with syringes, and the thread for that lock is shown more clearly in FIG. 13. The needle seat 30 is selectively moveable from the initial retained position to a needle retracted position (see FIG. 10), at which the attached needle 38 is substantially internal to the tubular body 14. The needle seat 30 includes a seat/plunger latch 32 (see FIGS. 6 and 13) opposite the end which receives the needle 38.

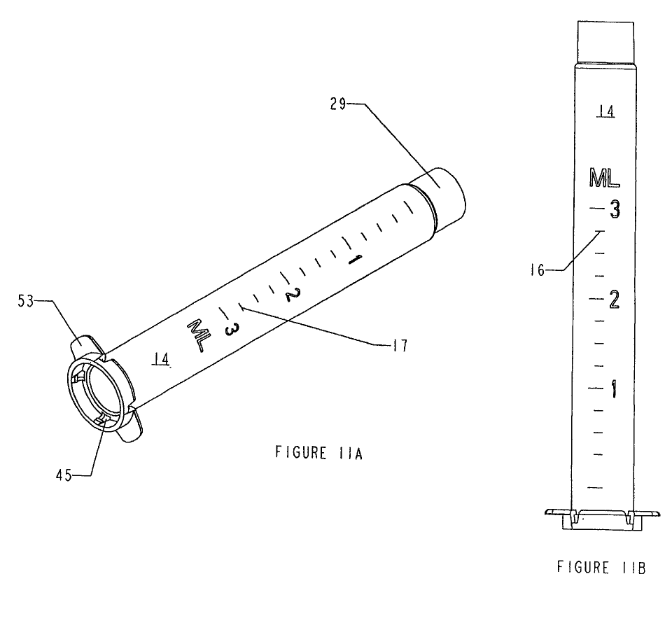

Referring again to FIG. 2, a plunger 42 may be used to expel the contents of the syringe 10 toward the needle end 18 and out through the needle 38. The plunger 42 has a needle seat end 46 positioned within the tubular body 14, and an opposed push plate end 50 extending from the tubular body 14, the plunger 42 being axially moveable relative to the tubular body 14. A rear plunger seal 44 is provided for sealing between the plunger 42 and the plunger end 22 of the tubular body 14. The plunger has a push plate 51 on the end 50, and the body 14 includes radially opposing finger tabs 53, as shown in FIGS. 2 and 11. The plunger 42 as shown in FIG. 2 has both a seat/plunger latch 54 and a plunger/stopper latch 58 near the needle seat end 46. As disclosed subsequently, the seat/plunger latch 54 on the plunger may selectively engage the seat/plunger latch 32 on the needle seat 30 to axially secure the needle seat 30 to the plunger 42. The plunger/stopper latch 58 may axially secure a stopper 62 to the plunger 42.

The initial packaged configuration of the syringe as shown in FIG. 1 is relatively compact, with the plunger 42 substantially within the tubular body 14. The needle seat end 46 is preferably near the needle seat 30, but the seat/plunger latch 54 is not yet engaged with the latch 32 on the needle seat 30. The stopper 62 is positioned near the plunger end 22, and is in sealed engagement with the tubular body 14.

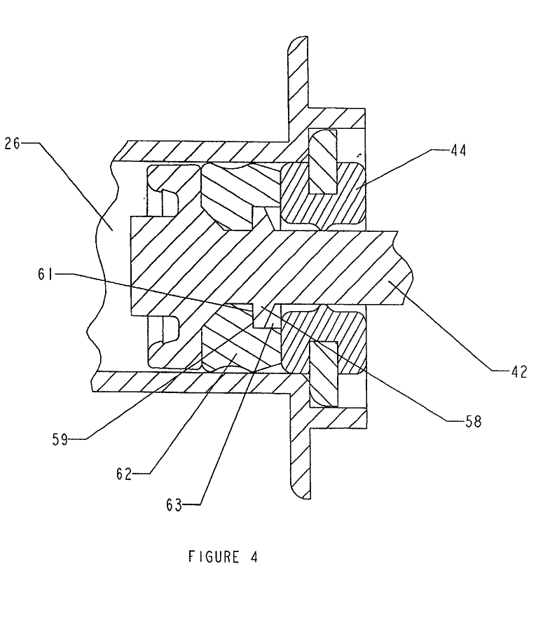

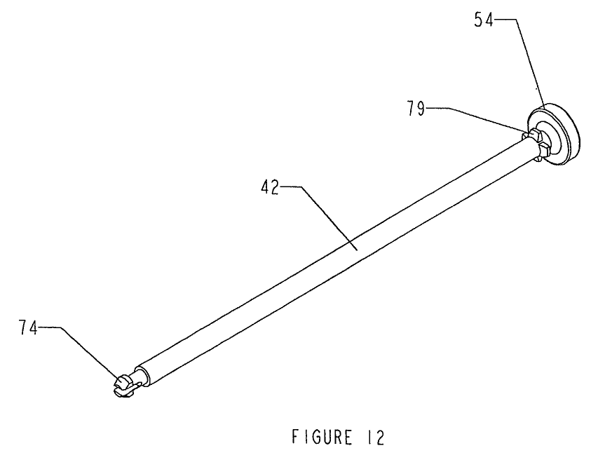

To initiate the filling process (see FIGS. 3 and 4), the plunger 42 is first moved into a fully retracted position to capture the stopper 62 via the plunger/stopper latch 58. During this process, the circumferentially spaced latch tabs 58 as shown in FIG. 12 thus compress the elastomeric material of stopper 62 against the bore walls of the throughbore 26, until the tabs 58 move into the stopper recess 63, with latch shoulder 59 on the plunger 42 engaging stop surface 61 on the stopper 62.

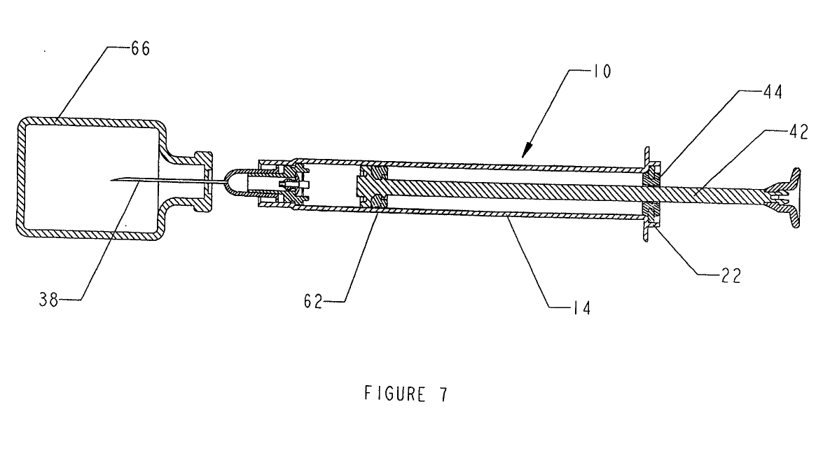

The plunger 42, along with the now captured stopper 62, may then be moved from an initial position to a selected displaced position, as shown in FIG. 7. Because the stopper 62 is sealingly engaged with the tubular body 14, pressure decreases within the tubular body 14 between the stopper 62 and the rear seal 44. Simultaneously, air in the syringe throughbore passes through the needle to pressurize the vial 66. A reverse gradation 16 as shown in FIG. 11 is preferably included on the tubular body 14 so the selected displaced position corresponds to a pre-determined volume of liquid that will be subsequently withdrawn from a fluid source 66.

The needle 38 is inserted or previously was inserted into the vial or other fluid source 66, and the plunger 42 is moved back toward the plunger end 22 of the tubular body 14 to a fluid withdrawn position, drawing a desired volume of fluid from the fluid source 66 into the syringe 10. The vacuum described above may assist the process of withdrawing fluid, although the valve described below preferably is biased closed with a sufficient force so that the healthcare practitioner pulls the plunger and thus the stopper toward the opposing plunger end of the body. Air may then be aspirated from within the body 14 through the needle 38. The syringe 10 and its contents are now ready for the injection process.

After the needle 38 has been inserted below the skin of a patient, the plunger 42 is forced axially toward the needle end 18, and the fluid forced by the stopper 62 out through the needle 38 and into the patient. At the end of the injection stroke, the seat/plunger latch 54 on the plunger 42 engages the seat/plunger latch 32 on the needle seat 30, thereby capturing the needle seat 30. In this fully stroked position, a substantial vacuum exists between the stopper 62 and the rear plunger seal 44, relative to the pressure in the tubular body 14 on the opposing side of the stopper 62. When the practitioner releases the axial force on the plunger 42, the created vacuum pulls the plunger 42 back toward the plunger end 22, disengaging the needle seat 30 from the tubular body 14, and withdrawing the needle seat 30 and attached needle 38 into a retracted position within the tubular body 14 (see FIG. 10). The generally annular bead 33 on the body 14 functions as a stop for the tabs 34 and thus the seat 30. The outer portion 34 initially engages this stop, but is subsequently moved radially inward by the latch 54, which both engages the seat via latch 32 and releases the seat 30 by disengaging tabs 34 from bead 33.

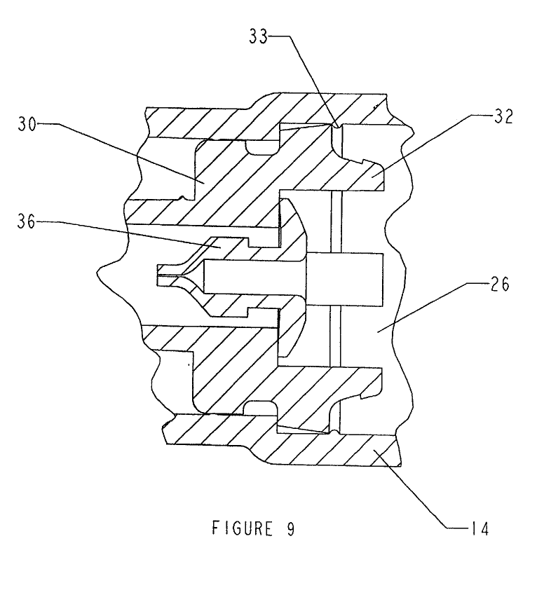

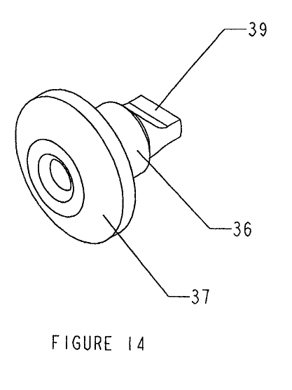

In a preferred embodiment, the syringe 10 includes a two-way valve 36 and as shown in FIGS. 6, 9 and 14 for governing fluid flow into and out of the tubular body 14. As shown in FIG. 6, the two-way normally closed valve 36 may include a first valve member 39 which is opened in response to relatively higher pressure internal to the tubular body 14 between the stopper 62 and the needle seat 30. The two-way valve 36 also includes a second valve member 37 which is opened in response to a relatively higher pressure external to the tubular body 14. The valve 39 may be referred to as a duck-bill valve, and the valve 37, which seals against planar surface 35 on the needle seat 30, may be referred to as an umbrella valve. When closed, the two-way valve 36 seals off needle seat 30, preventing liquid and/or air from entering or escaping the tubular body 14.

The two-way valve 36 may be formed from an elastomer so that it may be pressed into the central passageway 33 in the seat 30 (see FIG. 13), then locked in place about the reduced neck portion of the needle seat, with a pair of radially opposing ports as shown in FIG. 13 providing the flow channels to open the valve 37, releasing from a surface 65 of seat 30. The duck-bill valve 36 opens as shown in FIG. 6 to expel air or fluid from the body 14 through the needle. The inclusion of the two-way valve may also help control the movement of the plunger 42. For example, the two-way valve may prevent the plunger 42 from automatically retracting all the way to the plunger end 22 during the filling process. This may help control the operation of the syringe 10, for example, by preventing the plunger 42 from suddenly and unexpectedly snapping back the entire distance to the plunger end 22.

In a preferred embodiment, the seat/body retainer as shown in FIG. 6 may comprise a generally circumferential bead 33 on the body 14 and a plurality of circumferentially spaced locking tabs 34, each moveable radially inward in response to the seat/plunger latch 54. As the seat/plunger latch 54 engages with the seat/plunger latch 32 on the needle seat 30, the seat/plunger latch 54 also moves the locking tabs 34 radially inward, disengaging the locking tabs from the bead 33 on the tubular body 14, so that the needle seat 30 may freely retract into the tubular body 14, as described above.

Preferably the syringe according to the present invention is a disposable single-use device. The stopper is preferably sealingly secured to the plunger when the stopper/plunger latch 58 is engaged. The stopper/plunger latch preferably includes tabs extending radially outward of the cylindrical surface of the plunger for capturing the stopper. As explained herein, the plunger is axially connected to the stopper using the stopper/plunger latch, although in other embodiments the stopper could be axially secured to the plunger and the syringe shipped in that configuration to the practitioner. Since the plunger would extend axially a substantial distance from the cylinder body, the length of the syringe is undesirably increased, creating further handling and storage problems.

The needle may be shipped separately from the syringe, and the needle then attached to the needle seat in a conventional manner. In other embodiments, the needle and needle seat could be formed as a single unit, and the syringe shipped with the needle extending from the tubular body.

A preferred plunger/seat latch according to the present invention may include a female latch member 54 on a plunger and a male latch member 32 on the seat. The stopper/plunger latch includes a male member 58 on the plunger, with the body of a plunger serving as the female member. In other embodiments, the latch member may be provided on only one of the plunger and the seat for selectively securing the plunger to a seat, and a latch similarly may be provided on only one of the plunger and the stopper to selectively connect these components.

Various types of rear plunger seals may be used for reliably sealing between the rear portions of the tubular body and the plunger, and the seal 44 is merely exemplary. Various types of plungers or pistons may be used for reliably sealing the tubular body during axial movement of the plunger. Other types of retainers may be used for initially retaining the seat 30 in the needle end of the tubular body, and thereafter yielding or releasing the seat once engaged by the plunger to withdraw the needle seat and needle into the tubular body.

The method of the present invention will be readily understood to those skilled in the art in view of the above discussion. The syringe when used has the needle seat sealingly engaging the generally tubular body and retained in an initial retained position. The stopper is provided within the throughbore of a tubular body, and a plunger extends at least partially within the throughbore of the tubular body. The rear plunger seal is sealingly engaged with the plunger end of the tubular body and the plunger for effecting a vacuum chamber inside the tubular body and between the stopper and the rear plunger seal. A practitioner may axially interconnect the plunger and the stopper, then apply a selective first axial force to move the stopper from an initial position toward the opposing plunger end of the tubular body to a displaced position, thereby creating a partial vacuum within the tubular body. A needle is then inserted into the liquid source, but preferably is inserted prior to applying the first axial force to pressurize the liquid source (typically a vial) with air. A selected volume of liquid from the liquid source is then withdrawn into the tubular body between the needle seat and the stopper while simultaneously moving the stopper from a selected displaced position to a fluid drawn position. The biasing force on the closed valve 36 typically is greater than the vacuum force, so that the practitioner normally pulls the plunger back to draw fluid into the syringe. After moving the needle from the liquid source and aspirating the air from the syringe, the needle may be inserted into a fluid repository, such as the patient. Thereafter, a second axial force is applied by the practitioner to the plunger to move the stopper toward the needle end of the tubular body to discharge liquid from the tubular body and simultaneously create a partial vacuum within the tubular body between the stopper and the rear plunger seal. At the end of the injection stroke, the seat/plunger latch is engaged to connect the needle seat with one of the plunger and the stopper. The second force is then relaxed to disengage the needle seat from the tubular body and move the plunger and connected needle seat from the initial retained position to the needle seat retracted position.

In a preferred embodiment, the seat/plunger latch connects the needle seat with the plunger. In an alternate embodiment, the plunger may be provided with a latch mechanism for engaging the needle seat, in which case the seat may be connected to the plunger through the stopper.

The two-way normally closed valve is provided for withdrawing fluid into the tubular body and subsequently expelling the fluid from the tubular body. The syringe preferably includes graduations on the tubular body which increase from the plunger end toward the needle end. These graduations allow the practitioner to withdraw a selected quantity of liquid into the syringe.

A particular feature of the present invention is that the syringe is of relatively low cost, and the needle is reliably drawn into the tubular body after use. The syringe according to the present invention is particularly well suited for use when injecting a small quantity of fluid, e.g., less than 5 cc, and in some applications the tubular body may hold less than a maximum of about 3 cc.

In addition to medical applications, the syringe may be used in other non-medical applications. For example, if using the syringe for chemical extraction and disposal, rather than injection into a patient, the syringe 10 may be used to inject fluid into a different type of fluid receptacle, such as an open flask or other chemical handling media. The needle 38 may be designed accordingly to accommodate the desired process. For example, whereas a human patient may require use of a narrow, sharp needle, chemical extraction and disposal may require a larger needle to extract larger volumes of chemical fluids, or fluids with a higher viscosity.

It may be appreciated that changes to the details of the illustrated embodiments and systems disclosed are possible without departing from the spirit of the invention. While preferred and alternative embodiments of the present invention have been described in detail, it is apparent that further modifications and adaptations of the preferred and alternative embodiments may occur to those skilled in the art. However, it is to be expressly understood that such modifications and adaptations are within the spirit and scope of the present invention, as set forth in the following claims.

What is claimed is:

1. A syringe for retracting a needle supported on a needle seat, comprising:
a generally tubular body having a needle end and an opposing plunger end, the tubular body having a central axis and an internal throughbore extending between the needle end and the opposing plunger end, the tubular body receiving the needle seat therein and having a seat/body retainer at the needle end for axially initially attaching the needle seat to the generally tubular body;
the needle seat supported on and sealed to the tubular body by the seat/body retainer when the needle seat is in an initial retained position, and the needle seat being selectively moveable from the initial retained position to a needle seat retracted position;
a plunger having a needle seat end positioned within the internal throughbore of the generally tubular body and an opposing push plate end positioned external of the generally tubular body, the plunger being axially moveable relative to the tubular body and within the throughbore of the tubular body;
a stopper selectively moveable between an initial position in the opposing plunger end of the tubular body and a displaced position, the stopper axially secured to the plunger by a plunger/stopper connector and moveable in sealed engagement within the generally tubular body when moved from the initial position to the displaced position;
a seat/plunger latch for selective engagement of the needle seat with one of the plunger and the stopper;
a rear plunger seal in sealing engagement between the opposing plunger end of the tubular body and the plunger for effecting a vacuum chamber inside of the tubular body between the stopper and the rear plunger seal; and
a two-way normally closed valve for withdrawing fluid into the tubular body and subsequently expelling fluid from the tubular body.

2. The syringe as defined in claim 1, wherein the plunger/stopper connector comprises:
a stopper/plunger latch for selectively axially securing the stopper to the plunger by axial movement of the plunger with respect to the tubular body.

3. The syringe as defined in claim 2, wherein the stopper is sealingly secured to the plunger when the stopper/plunger latch is engaged.

4. The syringe as defined in claim 2, wherein the stopper/plunger latch includes circumferentially spaced protrusions extending radially outward of a cylindrical surface of the plunger.

5. The syringe as defined in claim 1, wherein the two-way valve includes a first valve member which is opened in response to fluid pressure between the needle seat and the stopper for discharging fluid from the syringe, and a second valve member opened in response to a vacuum between the needle seat and the stopper for drawing liquid into the syringe.

6. The syringe as defined in claim 5, wherein the second valve member seals against the needle seat when closed.

7. The syringe as defined in claim 1, wherein the two-way valve is secured to the needle seat.

8. The syringe as defined in claim 1, further comprising:
graduations on the tubular body increasing from the plunger end toward the needle end.

9. The syringe as defined in claim 1, wherein the seat/body retainer includes a plurality of circumferentially spaced locking tabs each moveable radially inward in response to the seat/plunger latch to release from stops on the generally tubular body.

10. The syringe as defined in claim 1, further comprising:
a push plate secured to the push plate end of the plunger for applying an axial force to the plunger.

11. The syringe as defined in claim 2, wherein:
the plunger/stopper latch includes one of a female connector and a male connector secured to the plunger and the other of the female connector and the male connector secured to the stopper; and
the seat/plunger latch includes one of a male connector and a female connector secured to the needle seat and the other of the male connector and the female connector secured to the plunger.

12. A method of using a syringe with a needle seat supporting a needle and sealingly engaged with and retractable into a generally tubular body having an internal throughbore extending between a needle end and an opposing plunger end, the method comprising:
sealingly engaging the needle seat with the generally tubular body, the needle seat being selectively movable from an initial retained position to a needle seat retracted position;
releasably retaining the needle seat in the initial retained position;
positioning the stopper within the throughbore of the tubular body;
positioning the plunger at least partially within the throughbore of the tubular body;
sealingly engaging a rear plunger seal with the opposing plunger end of the tubular body and the plunger for effecting a vacuum chamber inside of the tubular body and between the stopper and the rear plunger seal;
axially connecting the plunger and the stopper;
selectively applying a first axial force to the plunger to move the stopper from an initial position in the opposing plunger end of the tubular body to a selected displaced position while the stopper remains in sealed engagement with the generally tubular body, thereby creating a vacuum within the tubular body between the stopper and the rear plunger seal;
inserting a needle into a liquid source;
withdrawing a selected volume of liquid from the liquid source through the needle and into the tubular body between the needle seat and the stopper and simultaneously moving the stopper from the selected displaced position to a fluid drawn position;
thereafter inserting the needle into a fluid repository;
thereafter applying a second axial force to the plunger to move the stopper toward the needle end of the tubular body while discharging liquid from within the tubular body and simultaneously creating a partial vacuum within the tubular body between the stopper and the rear plunger seal;
engaging a seat/plunger latch to axially connect the needle seat with one of the plunger and the stopper; and
relaxing the second force to disengage the needle seat from the generally tubular body and move the plunger and the connected needle seat from the initial retained position to the needle seat retracted position.

13. The method as defined in claim 12, further comprising:
moving the plunger to engage a plunger/stopper latch to axially connect the plunger and the stopper.

14. The method as defined in claim 12, further comprising:
releasably retaining the needle seat in the initial retained position with a plurality of circumferentially spaced seat/body tabs on the seat and corresponding stops on the body.

15. The method as defined in claim 12, wherein the needle is inserted into the liquid source before moving the plunger to the displaced position, thereby pressurizing the liquid source while the plunger moves to the displaced position.

16. The method as defined in claim 12, further comprising:
providing a two-way normally closed valve for withdrawing fluid into the tubular body and subsequently expelling fluid from the tubular body.

17. A method of using a syringe with a needle sealingly supported on a needle seat, the syringe including a tubular body having an internal throughbore, the method comprising:
sealingly engaging the needle seat with the generally tubular body, the needle seat being selectively moveable from an initial retained position to a needle seat retracted position;
positioning a stopper within the tubular body, the stopper being in sealed engagement with the tubular body;
positioning a plunger at least partially within the throughbore of the tubular body;
sealingly engaging a rear plunger seal with a plunger end of the tubular body and the plunger for effecting a vacuum chamber inside of the cylinder body between the stopper and the rear plunger seal;
releasably retaining the needle seat in the initial retained position;
moving the plunger to connect the plunger with the stopper;
thereafter applying a first force to the plunger to move the stopper from an initial position to a selected displaced position, thereby creating a vacuum within the cylinder body between the stopper and the rear plunger seal;
withdrawing a volume of fluid from a fluid source through the needle and into the cylinder body between the needle seat and the stopper while simultaneously moving the stopper from the selected displaced position to a fluid drawn position;
thereafter aspirating air from within the cylinder body through the needle;
thereafter inserting the needle into a fluid repository;
thereafter applying a second force to the plunger for discharging fluid from within the cylinder body through the needle while creating a vacuum within the cylinder body between the stopper and the rear plunger seal;
engaging a seat/plunger latch to axially connect the needle seat with the plunger; and
relaxing the second force to disengage the needle seat from the generally tubular body and move the needle seat and the supported needle to a needle seat retracted position.

18. The method as defined in claim 17, further comprising:
releasably retaining the needle seat in the initial retained position with a plurality of circumferentially spaced seat/body tabs on the seat and corresponding stops on the body.

19. The method as defined in claim 17, wherein the needle is inserted into the liquid source before moving the plunger to the displaced position, thereby pressurizing the liquid source while the plunger moves to the displaced position.

20. The method as defined in claim 17, further comprising:
providing graduations on the tubular body increasing from the plunger end toward the needle end.
